(19) United States
(12) Patent Application Publication
(10) Pub. No.: US 2026/0363786 A1
(43) Pub. Date: Nov. 19, 2026

(54) INTELLIGENT SURGICAL INSTRUMENT WITH A COMBINATION METHOD FOR STAPLING LINE DETECTION AND LABELLING

(71) Applicant: GENESIS MEDTECH INTERNATIONAL PRIVATE LIMITED, Singapore (SG)

(72) Inventors: Shan Wan, Plymouth, MN (US); Bin ZHAO, Ellicott City, MD (US); Ning Li, Pittsburgh, PA (US)

(73) Assignee: GENESIS MEDTECH INTERNATIONAL PRIVATE LIMITED, (omitted)

(21) Appl. No.: 19/135,374

(22) PCT Filed: Dec. 4, 2023
(86) PCT No.: PCT/US2023/082239

§ 371 Date: Jun. 4, 2025

Related U.S. Application Data
(60) Provisional application No. 63/385,991, filed on Dec. 4, 2022.

(51) Int. Cl. A61B 34/20; A61B 1/313; A61B 17/00; A61B 17/072; G06T 7/00; G06T 7/246; G06V 10/14; G06V 10/764; G06V 10/82; G06V 20/40

(52) U.S. Cl. CPC A61B 34/20; A61B 17/072; G06T 7/0012; G06T 7/246; G06V 10/14; G06V 10/764; G06V 10/82; G06V 20/41; A61B 1/3132; A61B 2017/00119; A61B 2034/2065; G06T 2207/10016; G06T 2207/10068; G06T 2207/20084; G06T 2207/30004; G06V 2201/03

(57) ABSTRACT

A surgical instrument or system combining local sensing, computer vision and artificial intelligence algorithm to detect staple line, and its possible abnormality, and integrate it into laparoscopic or open surgical videos through AI data processing unit.

### CROSS-REFERENCE TO RELATED APPLICATIONS

This patent application claims the benefit of U.S. Provisional Patent Application No. 63/385,991, filed on Dec. 4, 2022, the entire content of which is incorporated as reference.

### BACKGROUND OF THE INVENTION

Surgical staplers have been used as instrumental tools for open and laparoscopic surgical procedures. Particularly since the 1980s laparoscopic video's commercialization, the endoscopic stapler/cutter has emerged as the minimally invasive solution for a wide spectrum of complex general/colorectal/GYN/bariatric/thoracic procedures. Mechanical surgical staplers have been open-loop devices till recent years when their feedback completely relied on surgeons' judgment and experience.

Staple line leak in surgery is a challenge, and it's more prevalent in sleeve gastrectomy. A properly formed B-shaped staple is correlated with a low leakage rate. One factor that may improve the B shape staple form is applying optimum pressure on the tissue at the stapling time. If the pressure is too high, which means the clamped tissue is too thick, the staple cannot close the loop to form the B-shape. If the pressure is too low, which means the clamped tissue is too thin, the grip would be low and cause tissue staking. The pressure on the tissue is dependent on the applied tissue pressure time since the tissue will dehydrate under pressure. As a result, quantifying the tissue pressure during clamping and firing may inform the decision-making process and improve the staple form. Another factor that may improve staple form is the proper usage of the stapler at the time of surgery. The proper use includes, but is not limited to, selecting the correct cartridge size and applying the optimal local shear forces to the tissue.

### SUMMARY OF THE INVENTION

Recent advances in surgical fields include adding power, basic and advanced sensing functions, and wireless data communication capabilities to the staplers. These additional features can be designed and implemented in the stapler, and connecting the stapler to a computation unit will extend it from a discrete device into a connected intelligent system. As a result, the system improves the ease of use and tool selection, and in certain circumstances, stapling consistency by understanding the force to close, force to fire, and dynamically provide force feedback.

Embodiments of this disclosure disclose an embedded sensor or an array of sensors at the end effector of the stapler, which can be embedded in the anvil or the cartridge. The sensors quantify the tissue pressure during clamping and firing, the data will be used in the further computation process and provide users with operation guidance to improve the form of the stapling line. The sensor data will also be used in calculating the local shear forces of the tissue caused by the applied force, and provide user guidance in selecting the correct cartridge size.

Embodiments of this disclosure disclose a surgical system including a surgical stapler with force detection and controlling function, and its interface with a data receiving and processing unit. The data receiving and processing unit can process the feedback data from the surgical system with real-time laparoscopic or open surgical video. As a result, an enhanced real-time video with a labeled risk zone can be displayed on the screen to provide information for operators.

### BRIEF DESCRIPTION OF THE DRAWINGS

FIG. 1 illustrates a firing force curve during a stapling process in a medical procedure.

FIG. 2a illustrates an exemplary image from a video showing a staple line.

FIG. 2b illustrates another exemplary image from an enhanced real time video showing a staple line.

FIG. 3a illustrates an exemplary console connecting a stapler, laparoscope, and monitor, according to an embodiment of the disclosure.

FIG. 3b illustrates the exemplary components of the console of FIG. 3a, according to an embodiment of the disclosure.

FIG. 4 is an exemplary image from the real live video that shows a detected staple line, according to an embodiment of the disclosure.

FIGS. 5a-5d illustrate images showing the recognition of staple line segments from images from real-time video of surgery, according to an embodiment of the disclosure.

FIG. 6 illustrates the exemplary hardware components of the console of FIG. 3b, according to an embodiment of the disclosure.

FIG. 7 is a flowchart illustrating the exemplary steps in the method of detecting staple lines during a medical procedure, according to an embodiment of the disclosure.

### DETAILED DESCRIPTION OF PREFERRED EMBODIMENTS OF THE INVENTION

Embodiments of the present disclosure will be described below in conjunction with the accompanying drawings, but it should be appreciated by those skilled in the art that the embodiments described below are exemplary, rather than exhaustive. They are only used to illustrate the present disclosure and should not be regarded as limiting the scope of the present disclosure. All other embodiments obtained by those of ordinary skill in the art without creative efforts based on the embodiments disclosed herein shall fall within the scope of the present disclosure.

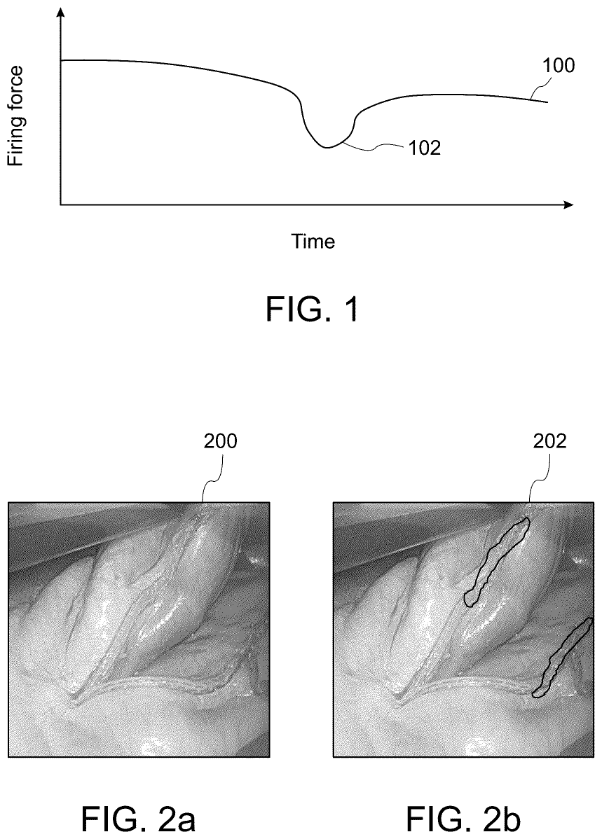

An example is shown in FIG. 1. The firing force curve 100 during a stapling process of a medical procedure is illustrated. It can be seen that during the stapling firing, there is a distinct drop of firing force 102 in this illustration. Although useful, it is very hard to interpret this information (i.e., the distinct drop of firing force) alone for clinical judgment. On the other hand, when combined with real-time video of the medical procedure, the imaging of the staple line, and its interaction with specific tissue can be used as input data. It is possible to determine the reason for the drop in firing force, which could be related to a lack of tissue clamped, missing staples, or tissue abnormality. In these cases, the video and data processing unit can calculate the abnormality parameters and fuse the real-time video with various labels, such as different colors or patterns.

FIG. 2b illustrates an example of part of staple line 202 being labeled in an enhanced real-time video, according to an embodiment of the disclosure. This will alert the surgeons to perform necessary surgical maneuvers and remedy if the surgeon deems the drop in firing force of the staple (and the effect it caused) as high risk. In contrast, FIG. 2a is an image showing staple line 200 from the real-time video of the operation without any labeling.

As an example of application, embodiments of the disclosed system can be used in bariatric or colorectal surgery where the system can label the staple lines with extreme tissue thickness, or firing over unwanted tissue where a higher leakage risk is present. A surgeon can further evaluate based on the alert (e.g., a labeled staple line section) to decide if further suturing or enhancement is needed.

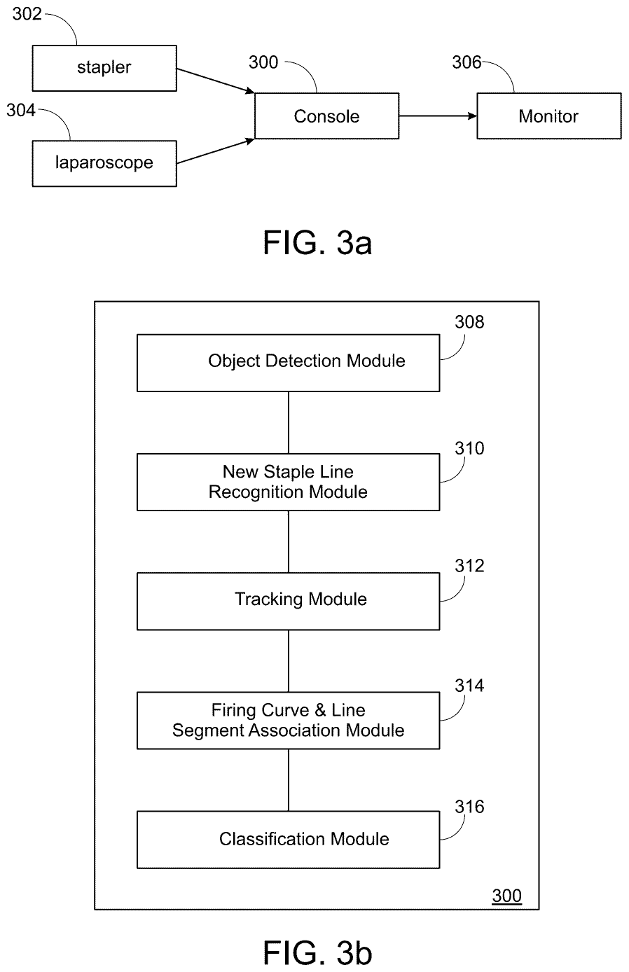

FIG. 3 illustrates an embodiment of the disclosed system. The system can include a console 300 connected to a stapler 302, a laparoscope 304, and a monitor 306. The connections between the console 300 and the other devices 302, 304, and 306 can be a wired or wireless connection, such as, but not limited to, a Wi-Fi connection, local area network (LAN) connection, or Bluetooth connection. The stapler 302 can transmit the firing force data of the stapler 302 to the console 300. The laparoscope 304 can transmit real-time video captured by the laparoscope 304 to the console 300. The console can process the firing force data and the real-time video and output alerts on monitor 306.

In one embodiment as illustrated in FIG. 3b, the console 300 can include a number of modules: an object detection module 308, a new staple line recognition module 310, a tracking module 312, a firing curve and staple line segment association module 314, and a classification module 316.

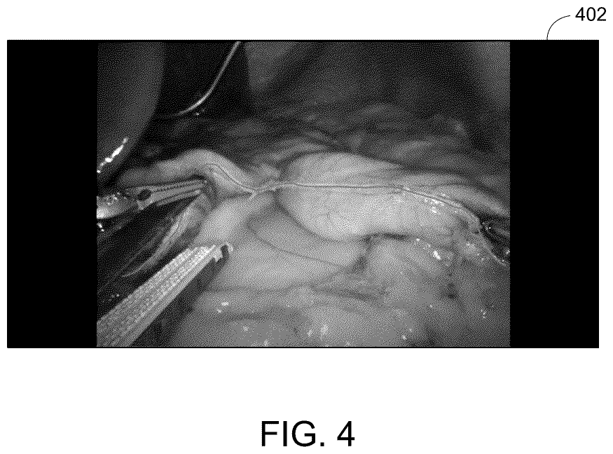

In this embodiment, the object detection module 308 can use a neural network (e.g., Region-based Convolutional Neural Networks (“RCNN) or You Only Look Once (“YOLO”)) to locate a staple line from an image. FIG. 4 provides an exemplary image from the real live video that shows in line 402 the detected staple line.

Multiple staple firings can happen in one surgery. The new staple line recognition module 310 can compare the detected staple line with previously detected staple lines to recognize a newly generated staple line. If it is the first firing of the stapler, the new staple line recognition module 310 can regard the entire staple line as a new staple line.

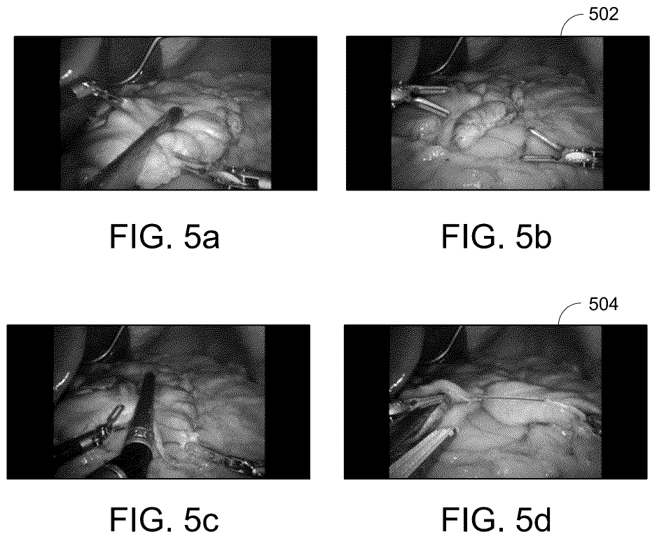

FIG. 5a is an image showing the first staple firing during surgery. FIG. 5b illustrates the new staple line 502 of the first staple firing of FIG. 5a as recognized by the new staple line recognition module 310. FIG. 5c is an image showing a second staple firing during the surgery. The new staple line recognition module 310 can compare the image of FIG. 5c with the image of FIG. 5a to recognize a newly generated second staple line 504 (shown in FIG. 4d).

Referring back to FIG. 3b, the tracking module 312 can utilize a neural network to track each staple line segment (each segment corresponds to an individual firing of the stapler). That is, the tracking module 312 can give the location of each staple line in every frame where the staple line is visible.

The firing curve and staple line segment association module 314 can associate the most recent staple firing curve with a new staple line segment once the new staple line segment is detected by tracking module 312. Such association matches each firing curve (e.g., the firing curve of FIG. 1) with a staple line segment (e.g., the staple line segments of FIG. 5b and FIG. 5d)

The classification module 316 can use a convolutional neural network (“CNN”) built to take the matched firing curve and staple line segment image as inputs and classify the input into one of several categories such as a) normal staple firing; b) abnormal staple firing with lack of tissue clamped; c) abnormal staple firing with possible missing staples; and d) abnormal staple firing with tissue abnormality.

The object detection module 308, new staple line recognition module 310, tracking module 312, firing curve and staple line segment association module 314, and classification module 316 of FIG. 3b can be implemented in software, firmware, hardware, or any combination of the three.

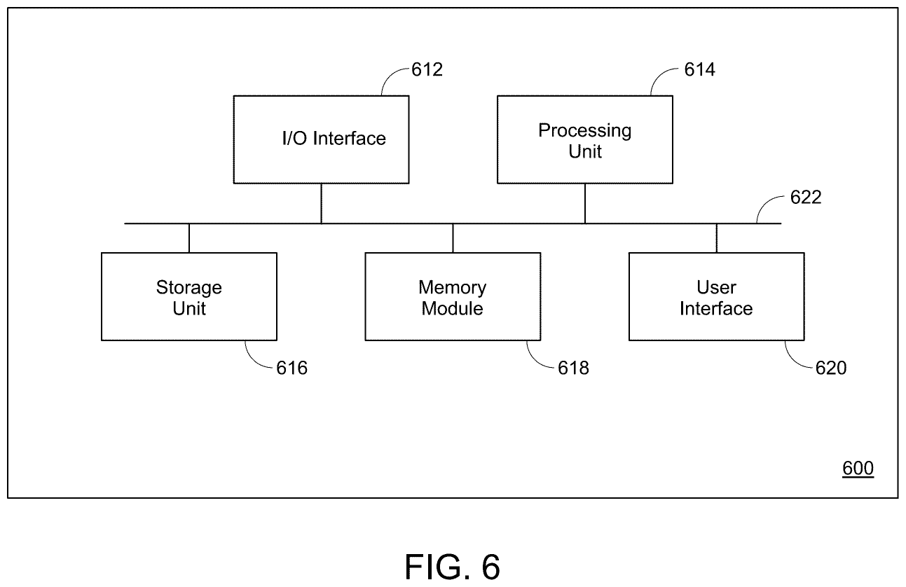

FIG. 6 illustrates the exemplary hardware components of the console of FIG. 3b, according to an embodiment of the disclosure. The console 600 can include, among other things, an I/O interface 612, a processing unit 614, a storage unit 616, a memory module 618, and a user interface 620, all connected via connection 622.

I/O interface 612 can be configured for communicating with a laparoscope and a stapler that are connected to the console 600. The I/O interface 612 can also communicate with a monitor to display alerts on the monitor. In some embodiments, separate I/O interfaces can be used for communicating with various devices, such as the laparoscope, the stapler, and the monitor. Communication can be via any suitable wired or wireless channel. Processing unit 614 may be configured to receive data from the laparoscope and the stapler and process the data to detect and label staple lines 600. Processing unit 614 may also be configured to generate and transmit signals, via I/O interface 612, to display alerts on the monitor.

Storage unit 616 and/or memory module 618 may be configured to store one or more computer programs that may be executed by processing unit 614 to perform functions of device 600. For example, the various exemplary modules of FIG. 3b can reside in storage unit 616 and/or memory module 618. Storage unit 616 and memory 618 can be non-transitory computer-readable medium storing instructions which, when executed, cause one or more processors 614 to perform the method, as discussed in the various embodiments of the disclosure.

The computer-readable medium can include volatile or non-volatile, magnetic, semiconductor, tape, optical, removable, non-removable, or other types of computer-readable medium or computer-readable storage devices. The computer-readable medium can have computer instructions stored thereon, as disclosed. In some embodiments, the computer-readable medium may be a disc or a flash drive having the computer instructions stored thereon.

It should be understood that console 600 of FIG. 6 can include additional components that are not shown in FIG. 6 and that some of the components shown in FIG. 6 may be optional in certain embodiments.

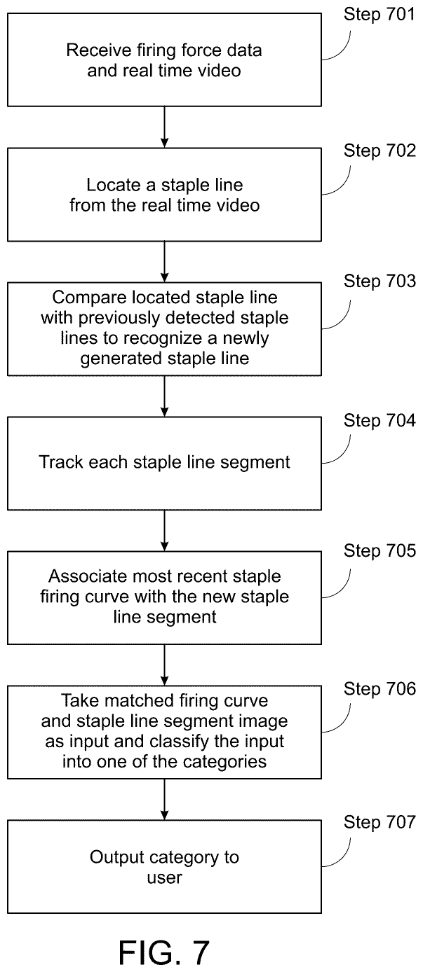

FIG. 7 is a flow chart illustrating the exemplary steps performed by the console 300 of FIG. 3. First, the console receives firing force data from a stapler and real-time video captured by a laparoscope during a medical procedure, such as surgery (Step 701). Next, the console can locate a staple line from an image from the real-time video (Step 702). This can be done using a neural network. The located staple line can be compared with previously detected staple lines to recognize a newly generated staple line (Step 703). If it is the first firing of the stapler, the entire staple line can be regarded as a new staple line.

The console can then track each staple line segment (each segment corresponds to an individual firing of the stapler) using a neural network (Step 704). That is, the console can determine the location of each staple line in every frame of the real-time video where the staple line is visible. The console associates the most recent staple firing curve generated from the firing force data received from the stapler with the new staple line segment once the new staple line segment is determined (Step 705). Essentially, each firing curve of the stapler is associated with a staple line segment identified from the real-time video.

The console may use a convolutional neural network (“CNN”) built to take the matched firing curve and staple line segment image as inputs and classify the input into one of several categories such as a) normal staple firing; b) abnormal staple firing with lack of tissue clamped; c) abnormal staple firing with possible missing staples; and d) abnormal staple firing with tissue abnormality. (Step 706) The category can be output to the user (Step 707).

The console may also record and classify the visual properties of the clamping tissue, the anvil location, and the cartridge size. The visual properties include organ/tissue classification, shape, abnormality, relative thickness, color, relative color variation, and surface vein structure. This data will be used to train a machine learning model that can predict the pressure changes based on the tissue and recommend appropriate cartridge size for that specific tissue and anvil location.

The console may also collect pressure sensor data and compare it to preset threshold values during the clamping process. The clamping process includes two phases: closing the jaw and resting period. Average tissue thickness is highly correlated with the maximum pressure applied on the tissue during clamping. If the max pressure is too high, it implies that tissue is relatively thick for that cartridge; if the max pressure is too low, it implies that tissue is relatively thin for that cartridge, in either case, the system will recommend a cartridge change.

The console may also collect and analyze the shear force on the tissue to recommend the stapler repositioning based on the variation in the pressure on the cartridge. One reason for not having a proper B-shaped staple form is the human factor. Doctors may apply unnecessary strains or tension on the tissue when applying the staple. If the pressure distribution is not uniform, the intelligent system may recommend stapler repositioning, including but not limited to rotation or translation, to minimize the pressure distribution variation to achieve a better stapler form.

A surgical stapler may be commercialized independently or in combination with an artificial intelligence (“AI”) processing unit and imaging system based on embodiments of this disclosure.

Although embodiments of this disclosure have been fully described with reference to the accompanying drawings, it is to be noted that various changes and modifications will become apparent to those skilled in the art. Such changes and modifications are to be understood as being included within the scope of embodiments of this disclosure as defined by the appended claims.

What is claimed is:

1. A system for stapling line detection during a medical procedure, the system comprising:
an object detection module configured to detect a staple line from an image;
a new staple line recognition module configured to compare the detected staple line with a previously detected staple line to recognize a newly generated staple line;
a tracking module configured to track each staple line segment, each staple line segment corresponding to an individual firing of a stapler;
a firing curve and staple line segment association module configured to associate a most recent staple firing curve with a new staple line segment after the new staple line segment is detected by the tracking module; and
a classification module configured to take the associated staple firing curve and new staple line segment as input and classify the input into one of a plurality of categories.

2. The system of claim 1, wherein the object detection module is configured to use a neural network to locate the staple line from the image.

3. The system of claim 2, wherein the neural network comprises either a Region-based Convolutional Neural Networks (“RCNN) or You Only Look Once (“YOLO”) Neural Network.

4. The system of claim 1, wherein the new staple line recognition module is configured to regard the detected staple line as a new staple line if the detected staple line is from a first firing of a stapler.

5. The system of claim 1, wherein the tracking module uses a neural network to track each staple line segment.

6. The system of claim 1, wherein the classification module uses a convolutional neural network (“CNN”) built to take the associated staple firing curve and new staple line segment as input and classify the input into one of a plurality of categories.

7. The system of claim 1, wherein the plurality of categories comprises (a) a normal staple firing, (b) an abnormal staple firing with lack of tissue clamped, (c) an abnormal staple firing with possible missing staples, and (d) an abnormal staple firing with tissue abnormality.

8. The system of claim 1, wherein the system is connected to a stapler and configured to receive firing force data of the stapler.

9. The system of claim 1, wherein the system is connected to a laparoscope and configured to receive a real-time video captured by the laparoscope during the medical procedure.

10. The system of claim 1, wherein the system is connected to a monitor and configured to output an alert on the monitor, and the alert is generated based on the category of which the input is classified.

11. A computer-implemented method of identifying a staple line, comprising:
detecting a staple line from an image;
comparing the detected staple line with a previously detected staple line to recognize a newly generated staple line;
tracking each staple line segment, each staple line segment corresponding to an individual firing of a stapler;
associating a most recent staple firing curve with a new staple line segment after the new staple line segment is detected by the tracking module; and
taking the associated staple firing curve and new staple line segment as input and classify the input into one of a plurality of categories.

12. The computer-implemented method of claim 11, further comprising using a neural network to locate the staple line from the image.

13. The computer-implemented method of claim 12, wherein the neural network comprises either a Region-based Convolutional Neural Network (“RCNN) or You Only Look Once (“YOLO”) Neural Network.

14. The computer-implemented method of claim 11, wherein comparing the detected staple line with a previously detected staple line to recognize a newly generated staple line comprises regarding the detected staple line as a new staple line if the detected staple line is from a first firing of a stapler.

15. The computer-implemented method of claim 11, wherein tracking each staple line segment comprises using a neural network to track each staple line segment.

16. The computer-implemented method of claim 11, wherein classify the input into one of a plurality of categories comprises using a convolutional neural network (“CNN”) built to take the associated staple firing curve and new staple line segment as input and classify the input into one of a plurality of categories.

17. The computer-implemented method of claim 11, wherein the plurality of categories comprises (a) a normal staple firing, (b) an abnormal staple firing with lack of tissue clamped, (c) an abnormal staple firing with possible missing staples, and (d) an abnormal staple firing with tissue abnormality.

18. The computer-implemented method of claim 11, further comprising connecting to a stapler and receiving firing force data of the stapler.

19. The computer-implemented method of claim 11, further comprising connecting to a laparoscope and receiving a real-time video captured by the laparoscope.

20. The computer-implemented method of claim 11, further comprising connecting to a monitor and outputting an alert on the monitor, the alert generated based on the category of which the input is classified.
